(19) United States
(12) Patent Application Publication
(10) Pub. No.: US 2003/0161892 A1
(43) Pub. Date: Aug. 28, 2003

(54) Topical clotting ointment and method

(76) Inventor: Connie L. McFarland, Deland, FL (US)

Correspondence Address: Connie McFarland, 630 South Hill Avenue, DeLand, FL, 32724, US

(21) Appl. No.: 10/085,460

(22) Filed: Feb. 27, 2002

(51) Int. Cl. A61K 35/78; A61K 33/32; A61K 31/718; A61K 31/704; A61K 31/366

(52) U.S. Cl. 424/642; 424/667; 424/756; 514/37; 514/57; 514/27; 424/727; 424/747; 424/765; 514/554; 514/460; 514/723

(57) ABSTRACT

A topical clotting ointment in which cumin in powder form or in oil form is a blood clotter in combination with an unctuous host substance and one or more infection-specific antiseptics and odorants. Method for manufacture includes processing for suspension of the cumin in the host substance and putting it preferably in a squeezable-tube dispenser (7) having an airtight cap (8).

### BACKGROUND OF THE INVENTION

This invention relates to topical salves for clotting blood at cuts, bleeding injuries, abrasions, lacerations and medical-needle perforations, including hemodialysis perforations.

Bleeding at medical-needle perforations, cuts and other skin openings does not always stop readily, particularly at re-openings, re-perforations of skin, abrasions and lacerations. There are numerous disinfectants, many of which are use related. However, there is no known clotting agent or ointment that utilizes cumin for health-care professional or patient use to stop bleeding quickly, easily and safely antiseptic in a manner taught by this invention.

Examples of most-closely related known but different medicinal substances and application methods are described in the following patent documents:

| U.S. Pat. No. | Inventor | Issue Date |
|---|---|---|
| 5,965,137 | Petrus | Oct. 12, 1999 |
| 4,254,145 | Birnbaum | Mar. 3, 1981 |
| 3,996,934 | Zaffaroni | Dec. 14, 1976 |
| 3,797,494 | Zaffaroni | Mar. 19, 1974 |
| 3,598,123 | Zaffaroni | Aug. 10, 1971 |
| 5,482,711 | Medenica | Jan. 09, 1996 |
| 4,737,360 | Allen, et al. | Apr. 12, 1988 |
| 5,997,889 | Durr, et al. | Dec. 7, 1999 |

### SUMMARY OF THE INVENTION

Objects of patentable novelty and utility taught by this invention are to provide a topical clotting ointment which:

stops bleeding quickly;

can be applied easily and conveniently at cuts, bleeding injuries, abrasions, lacerations and medical-needle perforations, including hemodialysis perforations; and

can be application-specific for particular types of infectious conditions.

This invention accomplishes these and other objectives with a topical clotting ointment in which cumin is a blood clotter in combination with an unctuous host substance for transmission, one or more infection-specific antiseptics and an optional odorant.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF DRAWINGS

This invention is described by appended claims in relation to description of a preferred embodiment with reference in part to the following drawings which are explained briefly as follows:

FIG. 1 is a list of ingredients by volume range of each for a general-purpose clotting ointment with powdered cumin;

FIG. 2 is a list of ingredients by volume range of each for a general-purpose clotting ointment with cumin oil;

FIG. 3 is a list of ingredients by volume range of each for a high-clotting ointment with powdered cumin;

FIG. 4 is a list of ingredients by volume range of each for a high-clotting ointment with cumin oil;

FIG. 5 is a list of ingredients by volume range of each for an infection-specific clotting ointment with powdered cumin;

FIG. 6 is a list of ingredients by volume range of each for an infection-specific clotting ointment with cumin oil;

FIG. 7 is a partially cutaway side view of a squeeze-tube ointment dispenser having an airtight cap with a taper-threaded seal;

FIG. 8 is a partially cutaway side view of a squeeze-tube ointment dispenser having an airtight cap with a straight-threaded seal; and

FIG. 9 is a flow diagram of a method for manufacturing the topical clotting ointment.

### DESCRIPTION OF PREFERRED EMBODIMENT

Listed numerically below with reference to the drawings are terms used to describe features of this invention. These terms and numbers assigned to them designate the same features throughout this description.

```
  1. General-purpose cumin-powder ointment
  2. General-purpose cumin-oil ointment
  3. High-clotting cumin-powder ointment
  4. High-clotting cummin-oil ointment
  5. Infection-specific cumin-powder ointment
  6. Infection-specific cumin-oil ointment
  7. Squeeze-tube dispenser
  8. Airtight cap
  9. Taper-threaded seal
10. Straight-threaded seal
11. Tapered nozzle
12. Non-tapered nozzle
13. Cumin preparation
14. Host selection
15. Selecting antiseptic
16. Odorant selection
17. Mixing ingredients
18. Forming unctuous consistency
19. Packaging for use
```

It has been known for centuries if not millenniums that cumin causes blood to clot. It was used commonly in straight powdered form during the Civil War to stop bleeding at abrasions and lacerations. However, there has been no medium or ointment invented for its effective application for either general-purpose, high-clotting or infection-specific use.

A host substance with unctuousness selected from a class of unctuous substances that include petroleum jelly, coconut butter, and suitably viscid vegetable oils has been found to be suitable for holding cumin in contact with blood proximate a skin perforation where it is applied. It has been found also that the cumin so held also causes the blood to clot in approximately one-fifth of the time as without it in most normal situations and faster yet in abnormal or aggravated situations where there has been prolonged perforation or repeated perforation of skin of a patient.

In conjunction with causing blood to clot for preventing bleeding, it has been found also that various types and proportions of antiseptic or disinfectants can be included. This allows preparation of salves and other treatments having particular levels of clotting characteristics as taught by this invention.

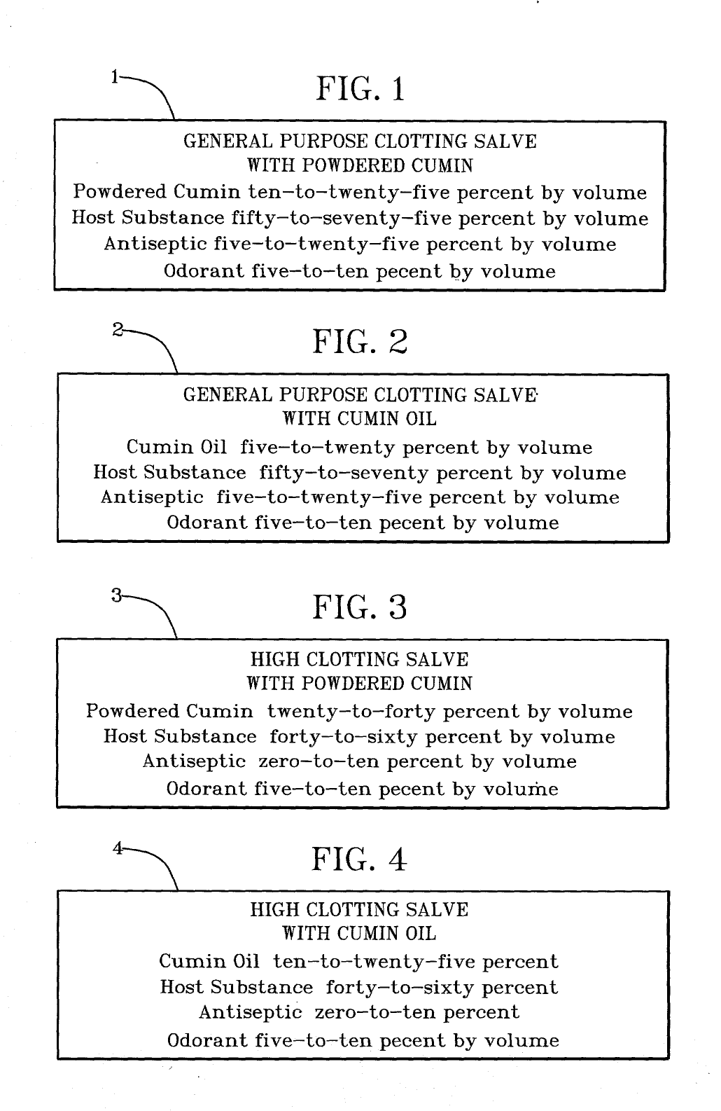

FIG. 1 lists ingredients by volume range of each for a general-purpose clotting ointment with powdered cumin. This is a general-purpose cumin-powder ointment 1 that can be applied to bleeding or potentially bleeding skin perforations as an antiseptic or disinfectant that also stops or prevents bleeding more effectively and more rapidly than other general-purpose salves or other treatment substances.

FIG. 2 lists ingredients by volume range of each for a general-purpose clotting ointment with cumin oil. This is a general-purpose cumin-oil ointment 2. Less cumin oil than cumin powder is required as shown. However, cumin oil is more expensive, especially during an introductory period prior to large-scale cultivation and processing. Either of these general-purpose clotting salves can be applied to bleeding or potentially bleeding skin perforations as an antiseptic or disinfectant that also stops or prevents bleeding more effectively and more rapidly than other general-purpose salves or other treatment substances.

FIG. 3 lists ingredients by volume range of each for a high-clotting ointment with powdered cumin. This is a high-clotting cumin-powder ointment 3. As shown, there is a greater proportion of cumin in proportion to host substance and antiseptic or disinfectant. This high-clotting ointment can be applied to bleeding or potentially bleeding skin perforations as an antiseptic or disinfectant that also stops or prevents bleeding more effectively and more rapidly than other salves or other treatment substances that require particularly high-clotting characteristics.

FIG. 4 lists ingredients by volume range of each for a high-clotting ointment with cumin oil. This is a high-clotting cumin-oil ointment 4. Less cumin oil than cumin powder is required as shown. However, as cumin oil is more expensive than cumin powder, it is recommended in accordance with cost-tradeoff factors. Either of these high-clotting salves can be applied to bleeding or potentially bleeding skin perforations as an antiseptic or disinfectant that also stops or prevents bleeding more effectively and more rapidly than other high-clotting salves or other treatment substances.

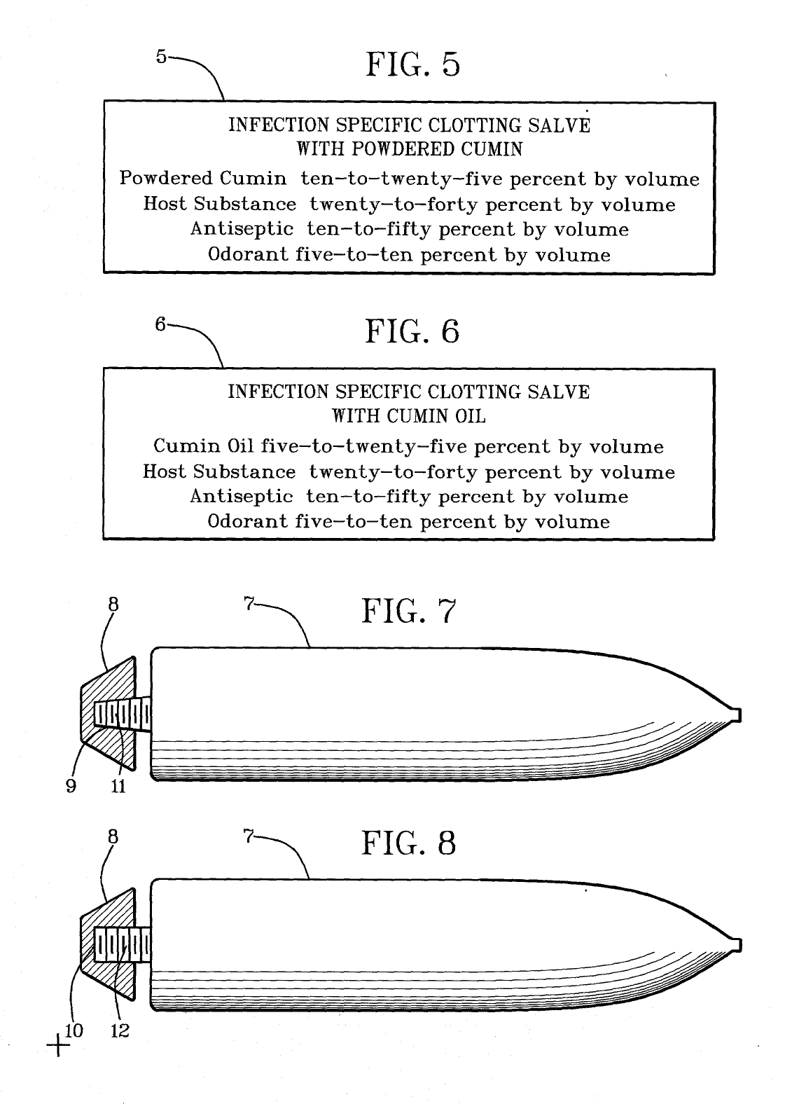

FIG. 5 lists ingredients by volume range of each for an infection-specific clotting ointment with powdered cumin. This is an infection-specific cumin-powder ointment 5. As shown, there is a greater proportion of antiseptic or disinfectant in proportion to cumin powder and in proportion to host substance. This infection-specific clotting ointment can be applied to bleeding or potentially bleeding skin perforations requiring particularly great antiseptic or disinfectant characteristics and also stops or prevents bleeding more effectively and more rapidly than other salves or other treatment substances that require particularly antiseptic or disinfectant characteristics.

FIG. 6 lists ingredients by volume range of each for an infection-specific ointment with cumin oil. This is an infection-specific cumin-oil ointment 6. Less cumin oil than cumin powder is required as shown. Again, however, as cumin oil is more expensive than cumin powder, it is recommended in accordance with cost-tradeoff factors. Either of these high-clotting salves can be applied to bleeding or potentially bleeding skin perforations as a particularly high antiseptic or disinfectant that also stops or prevents bleeding more effectively and more rapidly than other highly antiseptic and disinfectant salves or other treatment substances.

FIG. 7 depicts a squeeze-tube ointment dispenser 7 having an airtight cap 8 with a taper-threaded seal 9. Airtightness is more important for some forms of this topical clotting ointment having minute air bubbles and/or minute water particles. Manufacturing this topical clotting ointment containing minute air bubbles and/or minute water particles can be more expensive for highly stable consistencies than for less stable consistencies. Consistencies not having the minute air bubbles and/or water particles can be produced most inexpensively. For the least expensive consistencies without minute air bubbles and/or minute water particles and for the more expensive consistencies with the minute air bubbles and/or minute water particles, airtightness of the airtight cap 8 is no more critical than for conventional ointment dispensers. For least-expensive consistencies having minute air bubbles and/or minute water particles, however, airtightness of the airtight cap 8 is important for long shelf life.

FIG. 8 depicts a partially cutaway side view of the squeeze-tube ointment dispenser 7 for which the airtight cap 8 has a straight-threaded seal 10. The taper-threaded seal 9 of FIG. 7 seals at threads of a tapered nozzle 11 like pipe threading. The straight-threaded seal 10 of FIG. 8 seals at a cap-buttress end of a non-tapered nozzle 12.

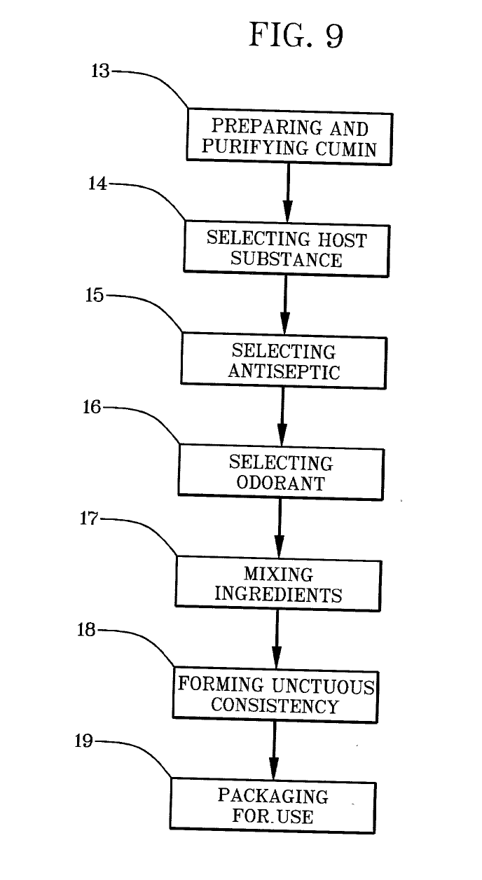

Referring to FIG. 9, cumin preparation 13 includes selection and purification of either cumin powder or cumin oil for a desired embodiment of the topical clotting ointment. This can be a cost-effectiveness selection of powder that is least expensive or oil which requires less. Purification can be included in the cost of preparing the cumin oil. Microwave energy or liquid disinfecting and drying of powdered cumin is related directly to prevailing costs of each.

Host selection 14 includes selection of a host material having unctuousness for achieving a desired hosting-conveyance objective for positioning the cumin predeterminedly in proximity to a skin perforation of a patient. This can include selection of petroleum jelly, viscid vegetable oil, coconut butter or other unctuous material. It includes optional selection for providing predetermined unctuousness in combination with minute particles of water and/or minute air bubbles when whipped.

Selecting antiseptic 15 includes selection for germicidal characteristics for achieving at least one predetermined antiseptic objective. This can include selection of either a known germicidal compound or separate germicidal substance. Selection is preferably from a class of antiseptics that include zinc salts, Bacitracin®, Neomycin®, Polymyxin B Sulfate®, Providone® iodine, Chlorhexidine Gluconate®, Methylparaben®, Glucono Delta Lactone®, glycerin, hydroxyethylcellulose, sodium hydroxide, Peg-8®, and Peg-75®. The antiseptic can be omitted if the host substance has sufficient antiseptic characteristics.

Odorant selection 16 includes selection for a predetermined odor objective. Cumin having a rather strong spice odor, it can be mixed well with a class of odorous substances that include vanilla extract, anise powder, peppermint extract, menthol, rose oil, jasmine oil, pine oil, lemon oil and coconut oil. The odorant can be optional, depending on odors and odor-suppressing effects of the other ingredients.

Mixing ingredients 17 includes mixing the cumin, the host substance, the antiseptic if used and the odorant if used.

Forming an unctuous consistency 18 can include whipping, homogenizing of other process to achieve a predetermined unctuousness in comparison to liquidity for desired uses. Some uses will benefit from rigidity of unctuousness. Others will benefit from more liquidity of unctuousness.

Packaging for use 19 for some of the less-stable consistencies is best performed immediately after a predetermined consistency is achieved. Other more-stable consistencies can be bulk-packaged for later packaging for use 19 by large-volume users that include hospitals, nursing homes, universities and clinics.

A new and useful topical clotting ointment having been described, all such foreseeable modifications, adaptations, substitutions of equivalents, mathematical possibilities of combinations of parts, pluralities of parts, applications and forms thereof as described by the following claims and not precluded by prior art are included in this invention.

What is claimed is:

1. A topical clotting ointment comprising:
an effective amount by volume of powdered cumin; and
an effective amount by volume of one or more host substances selected from a class of unctuous substances that include petroleum jelly, coconut butter and selectively viscid vegetable oil.

2. The topical clotting ointment of claim 1 further comprising:
an effective amount by volume of one or more antiseptics selected from a class of antiseptics that include zinc salts, Bacitracin®, Neomycin®, Polymyxin B Sulfate®, Providone® iodine, Chlorhexidine Gluconate®, Methylparaben®, Glucono Delta Lactone®, glycerin, hydroxyethylcellulose, sodium hydroxide, Peg-8®, and Peg-75®.

3. The topical clotting ointment of claim 1 further comprising:
an effective amount by volume of one or more odorants selected from a class of odorous substances that include vanilla extract, anise powder, peppermint extract, menthol, rose oil, jasmine oil, pine oil, lemon oil and coconut oil.

4. The topical clotting ointment of claim 2 and further comprising:
an effective amount by volume of one or more odorants selected from a class of odorous substances that include vanilla extract, anise powder, peppermint extract, menthol, rose oil, jasmine oil, pine oil, lemon oil and coconut oil.

5. The topical clotting ointment of claim 1 wherein:
the topical clotting ointment is a general-purpose clotting ointment in which the effective amount of powdered cumin is ten-to-twenty-five percent by volume; and
the effective amount of host substance is fifty-to-seventy percent by volume.

6. The topical clotting ointment of claim 2 wherein:
the effective amount of antiseptic is five-to-twenty-five percent by volume.

7. The topical clotting ointment of claim 1 wherein:
the host substance contains minute air bubbles for allowing penetration of blood for mixture with the powdered cumin proximate a perforated-skin area of a patient.

8. The topical clotting ointment of claim 7 wherein:
the host substance contains minute water particles for mixture of blood with the powdered cumin proximate a perforated-skin area of a patient.

9. The topical clotting ointment of claim 1 wherein:
the host substance contains minute water particles for mixture of blood with the powdered cumin proximate a perforated-skin area of a patient.

10. The topical clotting ointment of claim 1 wherein:
the topical clotting ointment is a high-clotting ointment in which the effective amount by volume of powdered cumin is twenty-to-forty percent, the effective amount by volume of host substance is forty-to-sixty percent and the effective amount by volume of antiseptic is zero-to-ten percent.

11. The topical clotting ointment of claim 1 wherein:
the topical clotting ointment is an infection-specific clotting ointment in which the effective amount by volume of powdered cumin is ten-to-twenty-five percent, the effective amount by volume of host substance is twenty-to-forty percent and the effective amount by volume of the antiseptic which is infection-specific is ten-to-fifty percent.

12. A method for manufacture of a topical clotting ointment having an effective amount by volume of cumin and an effective amount by volume of one or more host substances selected from a class of unctuous substances that include petroleum jelly, coconut butter and selectively viscid vegetable oil; the method comprising the steps of:
purifying the cumin in a manner that does not destroy a blood-clotting characteristic which it contains;
selecting a host substance having unctuousness for achieving a desired hosting-conveyance objective for positioning the cumin in proximity to a skin perforation of a patient;
selecting a desired odorant;
mixing the cumin, the host substance, the antiseptic and the odorant; and
forming a predeterminedly unctuous consistency of a mix of the cumin, the host substance, the antiseptic and the odorant for the topical clotting ointment.

13. The method of claim 12 and further comprising:
putting the topical clotting ointment in a ointment dispenser for use by health-care givers and by patients.

14. The method of claim 12 wherein:
disinfecting the cumin is by application of microwave energy and then cooling.

15. The method of claim 12 wherein:
disinfecting the cumin is by soaking in a liquid germicide and then drying.

16. The method of claim 15 wherein:
the liquid germicide is alcohol.

17. The method of claim 12 wherein:
the host substance includes unctuousness that supports minute stabilized air bubbles by being whipped in mixture with the cumin and the antiseptic; and
whipping the mixture into a stabilized froth having a predetermined consistency.

18. The method of claim 17 wherein:
the host substance includes unctuousness that supports minute stabilized water particles by being whipped in mixture with the cumin and the antiseptic; and
whipping the mixture into a predeterminedly stabilized water-particle consistency.

19. The method of claim 12 wherein:
the host substance includes unctuousness that supports minute stabilized water particles by being whipped in mixture with the cumin and the antiseptic; and
whipping the mixture into a predeterminedly stabilized water-particle consistency.

20. The method of claim 12 wherein:
the ointment dispenser is a squeeze tube having an airtight cap on a dispenser nozzle.

21. The method of claim 20 wherein:
the airtight cap includes tapered threading on an inside periphery of an attachment sleeve of the airtight cap; and
the dispenser nozzle has an outside periphery that includes tapered threading which matches the tapered threading on the inside periphery of the attachment sleeve of the airtight cap.

22. The method of claim 20 wherein:
the airtight cap includes non-tapered straight threading on an inside periphery of an attachment sleeve of the airtight cap;
the dispenser nozzle has an outside periphery that includes non-tapered straight threading which matches the non-tapered straight threading on the inside periphery of the attachment sleeve of the airtight cap;
the airtight cap includes a circumferential sealing surface proximate an inside end of the attachment sleeve; and
the dispenser nozzle includes a circumferential sealing tip which buttresses against the circumferential sealing surface of the airtight cap.

23. A topical clotting ointment comprising:
an effective amount by volume of cumin oil; and
an effective amount by volume of one or more host substances selected from a class of unctuous substances that include petroleum jelly, coconut butter and selectively viscid vegetable oil.

24. The topical clotting ointment of claim 23 further comprising:
an effective amount by volume of one or more antiseptics selected from a class of antiseptics that include zinc salts, Bacitracin®, Neomycin®, Polymyxin B Sulfate®, Providone® iodine, Chlorhexidine Gluconate®, Methylparaben®, Glucono Delta Lactone®, glycerin, hydroxyethylcellulose, sodium hydroxide, Peg-8®, and Peg-75®.

25. The topical clotting ointment of claim 23 and further comprising:
an effective amount by volume of one or more odorants selected from a class of odorous substances that include vanilla extract, anise powder, peppermint extract, menthol, rose oil, jasmine oil, pine oil, lemon oil and coconut oil.

26. The topical clotting ointment of claim 23 wherein:
the topical clotting ointment is a general-purpose clotting ointment in which the effective amount of cumin oil is five-to-twenty percent by volume; and
the effective amount of host substance is fifty-to-seventy percent by volume.

27. The topical clotting ointment of claim 24 further comprising:
the effective amount of antiseptic is five-to-twenty-five percent by volume.

28. The topical clotting ointment of claim 23 wherein:
the host substance contains minute air bubbles for allowing penetration of blood for mixture with the cumin oil proximate a perforated-skin area of a patient.

29. The topical clotting ointment of claim 28 wherein:
the host substance contains minute water particles for mixture of blood with the cumin oil proximate a perforated-skin area of a patient.

30. The topical clotting ointment of claim 23 wherein:
the host substance contains minute water particles for mixture of blood with the cumin oil proximate a perforated-skin area of a patient.

31. The topical clotting ointment of claim 23 wherein:
the topical clotting ointment is a high-clotting ointment in which the effective amount by volume of cumin oil is ten-to-twenty-five percent, the effective amount by volume of host substance is forty-to-sixty percent and the effective amount by volume of antiseptic is zero-to-ten percent.

32. The topical clotting ointment of claim 23 wherein:
the topical clotting ointment is an infection-specific clotting ointment in which the effective amount by volume of cumin oil is five-to-twenty percent, the effective amount by volume of host substance is twenty-to-forty percent and the effective amount by volume of the antiseptic which is infection-specific is ten-to-fifty percent.
